(19) United States
(12) Patent Application Publication
(10) Pub. No.: US 2007/0265673 A1
(43) Pub. Date: Nov. 15, 2007

(54) FLEXIBLE INTERCONNECT ASSEMBLY FOR IMPLANTABLE MEDICAL DEVICES

(76) Inventors: Terrance Ransbury, Chapel Hill, NC (US); Stephen Purcell, Carborro, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/696,142

(22) Filed: Apr. 3, 2007

Related U.S. Application Data
(60) Provisional application No. 60/788,690, filed on Apr. 3, 2006.

(51) Int. Cl. A61N 1/00

(52) U.S. Cl. 607/36; 29/428

(57) ABSTRACT

A modular implantable medical device having first and second component containers and a flexible connector for connecting the first and second containers. The connector can include a pair of end members and a flexible portion extending therebetween to define an internal passage between the end members. Each of the end members can include a receptacle configured to communicate with the containers. The connector further includes a conduit extending between the pair of end members in the internal passage to provide a communication path between the first and second containers such that the first and second component containers are in communication.

### RELATED APPLICATION

The present application claims the benefit of U.S. Provisional Application No. 60/788,690, filed Apr. 3, 2006, which is incorporated herein in its entirety by reference.

### FIELD OF THE INVENTION

The present invention generally relates to implantable medical devices. More particularly, the present invention can relate to flexibly interconnecting functional components in implantable medical devices.

### BACKGROUND OF THE INVENTION

Devices such as pacemakers, defibrillators, and implanted cardioverter defibrillators (ICD) can be implanted to treat heart conditions. Specifically, pacemakers can be implanted to detect periods of bradycardia and deliver electrical stimuli to increase the heartbeat. ICDs can be implanted to cardiovert or defibrillate the heart by delivering electrical current directly to the heart. Another type of implantable defibrillation device can be used to detect an atrial fibrillation (AF) episode and deliver an electrical shock to the atria to restore electrical coordination.

Elongated forms of such implanted devices, e.g., pacemakers, defibrillators, drug pumps, or similar intravascular implantable devices, can contain multiple elongate rigid or semi-rigid containers having interior spaces therein for functional components, such as batteries, capacitors, microprocessors, and circuitry. Examples of such component containers are described, for example, in U.S. Pat. No. 7,082,336, entitled “Implantable Intravascular Device for Defibrillation and/or Pacing.” The components can be electrically connected using flex circuits and the component containers can be mechanically connected using a flexible material, such as silicone rubber filler, to form hinges that bend in response to passage of the device through curved regions of the vasculature. Further examples of containers and connectors for the electric components are described, for example, in U.S. Patent Publication No. 2006/0217779, entitled “Flexible Hermetic Enclosure for Implantable Medical Devices,” which describes using mechanical bellows for connecting containers.

Due to the length of a series of elongated component containers for implantable intravascular devices, such as described in the above-mentioned references, any electrical wires, cables, flex circuits, conduits, tubing, and other connectors often must be threaded through multiple segments of the device or over a longer cable, flex circuit, thread, or wire extending through the device. In most implantable pulse generators, the needs for maintaining a hermetic seal of any wires, tubes or other connections extending outside a given container have required the use of feed through arrangements, as described, for example, in U.S. Pat. Nos. 6,961,232, 6,505,073, 5,336,246 and 4,934,366, and U.S. Publ. Appl. No. 2004/0116976 A1. These existing feed through arrangements are typically between a single can or housing and an associated header.

Few, if any, implantable medical devices have needed multiple interconnections among articulating, elongated containers or housings. U.S. Pat. No. 6,254,626 describes a catheter device for heating or cooling organs that comprises an elongated device with articulated segments and flexible metal or rubber tubes between segments that includes an inner lumen capable of transporting a pressurized liquid to a distal end of the catheter. The catheter described in this patent, however, is for use in an acute procedure and is not adapted for chronic, long-term implantation. There is a therefore a current need for an implantable medical device addressing the above-referenced issues.

### SUMMARY OF THE INVENTION

The present invention overcomes the above referenced deficiencies by providing a device enabling assembly of functional components of elongated medical devices in a modular fashion.

A modular implantable medical device according to an embodiment can include first and second component containers, a flexible connector for connecting the first and second containers having a pair of end members and a flexible portion, such as a bellows, extending therebetween to define an internal passage between the end members. One of the end members can include a first receptacle configured to communicate with a portion of the first container and the other of the end members can include a second receptacle configured to communicate with a portion of the second container. The device can further include a conduit extending between the end members in the internal passage to provide a communication path between the first and second containers, such that when the first container is in communication with the first receptacle and the second container is in communication with the second receptacle, a portion of the first and second containers are in communication via the conduit.

A flexible connector for an elongate medical device according to an embodiment can include first and second end members, each of the end members formed of non-conductive material, a flexible portion extending between the end members defining an internal passage therebetween, and a conducive member presented in the internal passage extending between a first conductive region presented on the first end member and a second conductive region presented on the second end member to electrically connect the first and second conductive regions.

A method for connecting containers of an implantable medical device includes providing a first component container and a second component container, providing a connector including a first end member having a first receptacle therewith and a second end member having a second receptacle therewith and a flexible portion extending between the first and second end members to define a passage between the end members, wherein a conduit extends between the end members in the passage to provide a communication path between the first and second end members, and coupling the first component container with the first end member and the second component container with the second end member, such that the first and second component containers are in communication via the conduit.

### BRIEF DESCRIPTION OF THE DRAWINGS

FIG. 1 is a fragmentary elevational view of an implantable device depicting a first container, a connector, and a portion of a second container;

FIG. 2 is an elevational view illustrating the flexibility of the implantable device of FIG. 1;

FIG. 3 is an exploded elevational view of an embodiment of the connector of FIG. 1;

FIG. 4 is a perspective view of a disk or end portion as part of the connector of FIG. 3;

FIG. 5 is a perspective view of the connector system of FIG. 3, with bellows and collar depicted cut away to view internal components, such as conductors, therein;

FIG. 6 is an exploded perspective view of an embodiment of the container of FIG. 1;

FIG. 7 is a perspective view of another embodiment of a flex circuit of the container of FIG. 6;

FIG. 8 is an elevation view of the flex circuit of FIG. 7;

FIG. 8a is a close-up view of a first end of the flex circuit of FIG. 8;

FIG. 8b is a close-up view of a middle section of the flex circuit of FIG. 8;

FIG. 8c is a close-up view of a second end of the flex circuit of FIG. 8;

FIG. 9 depicts a container and connector prior to coupling thereof;

FIG. 10 is a cross-sectional elevational view of an implantable device having one container and two connectors;

FIG. 10a is a close-up view of a first connector-container connection of FIG. 10; and

FIG. 10b is a close-up view of a second connector-container connection of FIG. 10.

FIG. 11 is an isometric view of another embodiment of a connector-container arrangement.

FIGS. 12a-12e are more detailed views of the connector of FIG. 11.

### DETAILED DESCRIPTION OF THE DRAWINGS

There are a number of techniques for implanting an elongated, flexible medical device in a patient, such as in vasculature, as described in U.S. Patent Publication Nos. 2006/0217779 and 2005/0043765, both of which are incorporated herein by reference in their entirety. Examples of intravascular medical implants having electronic and electrical components housed within an elongate housing proportioned for placement in the vasculature are described, for example, in U.S. Pat. No. 7,082,336 and U.S. Patent Publication Nos. 2004/0249431 and 2005/0043765, all of which are incorporated herein by reference in their entirety.

The electronic and electrical components and circuitry within these devices can be hermetically sealed to inhibit damage and also inhibit the release of contaminants into the bloodstream. Further, due to the length of these implantable devices, these devices can be designed with sufficiently flexibility vis-a-vis container connectors to move through the vasculature while being sufficiently rigid to protect the internal components.

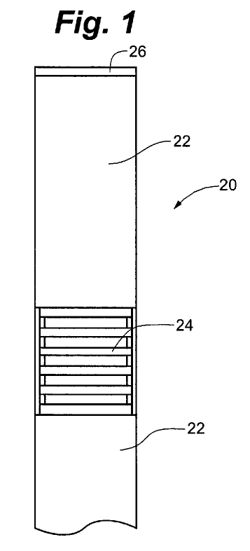

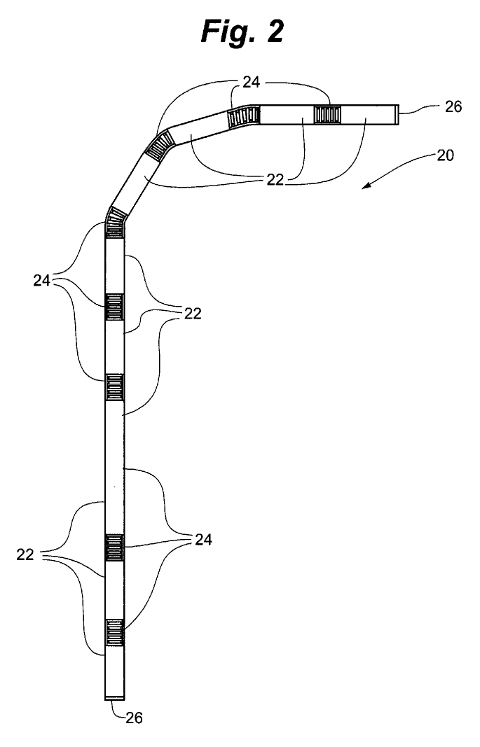

Referring to FIGS. 1 and 2, a modular implantable medical device 20 includes a plurality of multiple rigid or semi-rigid enclosures or containers 22 for containing electronic or other components necessary for the implantable device and one or more flexible connectors 24 for flexibly interconnecting containers 22. Connectors 24 can provide, for example, flexibility, columnar strength, and torqueability to device 20. Connectors 24 can be connected to containers 22 so as to form a continuous hermetic seal, such that standard wiring and components within device 20 can be used without fear of corrosion or contamination. One or more end caps 26 can be included on containers at the ends of device 20 to further provide the continuous hermetic seal.

Referring to FIG. 2, containers 22 can be connected using flexible connector 24, such as silicone rubber tubes or mechanical bellows, to form articulations. These articulations form hinges that bend in response to passage of device 20 through curved regions of the vasculature. Examples of mechanical bellows are described in U.S. Patent Publication No. 2006/0217779, which is incorporated herein by reference in its entirety.

As described above, containers 22 can house functional components that can be used to carry out the system functions of implant device 20. For example, containers 22 within implant device 20 can collectively include one or more pulse generators, including associated batteries, capacitors, microprocessors, and circuitry for generating electrophysiological pulses for defibrillation, cardioversion, and/or pacing. Containers 22 can also house detection circuitry for detecting arrhythmias or other abnormal activity of the heart. The specific components contained within containers 22 can depend upon the application for device 20, such as whether device 20 is intended to perform defibrillation, cardioversion, and/or pacing, in addition to sensing functions. If device 20 has drug delivery capability, the functional components can include fluid reservoirs and pumps in fluidic communication via tubular conduits. Other functional components can include those in optical communication with one another using optical cabling.

While containers 22 are depicted herein to have a cylindrical or tubular shape, containers 22 can be of any appropriate shape, cross-section, and length suitable for the application for device 20.

In the present application, containers 22 specifically have a cylindrical shape with a diameter of 3 mm to about 15 mm and an axial length of about 20 mm to about 75 mm. To enable insertion of the series of rigid or semi-rigid component containers 22 into the vasculature, it can be desirable to limit the diameter to less than about 8 mm with a length of less than about 70 cm. Given the minimal space for component within device 20, it can be desirable to arrange the device 20 components so as to make efficient use of the available space. The length of the components can vary depending upon the ultimate destination of each component and the path through which each component must pass, as the amount of bending and varying size of the path can affect the maximum component size for different areas of the vasculature. Additional diameter and length ranges within the explicit ranges given above are contemplated and are within the present disclosure.

The thickness of the walls of containers 22 also can vary, depending upon the application and the materials being used. Often, it can be desirable for the walls to be as light as possible, while still providing for sufficient rigidity. In one example, container 22 can be made of a biocompatible material that is capable of sterilization and is conductive, with a sidewall thickness of about 0.001″ to 0.005″. The sidewall thickness can vary between containers, as well as within an individual container in order to accommodate the internal components or the like. Additional ranges within the explicit range given above are contemplated and are within the present disclosure. Container 22 wall materials include titanium, nitinol, stainless steel, nickel, or alloys thereof, as well as polymers such as nylon or polyurethane.

Depending upon the material being used, containers 22 can be covered by a layer or coating or sleeve that can be electrically insulative, particularly if container 22 material is conductive. Examples of such coatings include but are not limited to polyurethanes, silicone, and ePTFE. It can be desirable to provide a coating that is anti-thrombogenic (e.g., perfluorocarbon coatings applied using supercritical carbon dioxide), so as to prevent thrombus formation on the device. It also can be beneficial for the coating to have anti-proliferative properties so as to minimize endothelialization or cellular ingrowth, as minimizing growth into or onto the device can help minimize vascular trauma when the device is explanted. The coating can also be selected to elute anti-thrombogenic compositions (e.g., heparin sulfate) and/or compositions that inhibit cellular in-growth and/or immunosuppressive agents. If container 22 is conductive, this layer or coating can be selectively applied or removed to leave an exposed electrode region on the surface of the enclosure where necessary.

Any appropriate number of containers 22 can be mechanically connected using connectors 24, such as interconnecting bellows, to form flexible implantable medical device 20. For many devices 20, this can include a string of at least three containers 22. The sequence of devices 20 and linking connectors 24 can be repeated as necessary to make device 20 of an appropriate length. For example, as depicted in FIG. 2, device 20 can include several containers 22 connected using multiple connectors 24 as needed to accommodate the components needed for performance of device 20. Device 20 can include other features such as stimulation leads positioned in a right ventricle (RV) of a human heart and anchors for retaining the device in a blood vessel, such as described in U.S. Patent Publication Nos. 2006/0217779 and U.S. Pat. No. 7,082,336, both of which are incorporated herein by reference in their entirety.

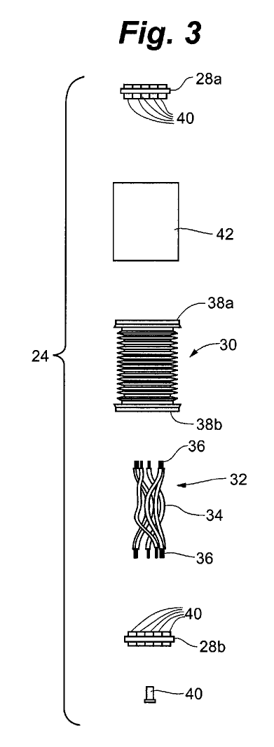

Referring to FIG. 3, connector 24 generally includes a pair of connection members or disks 28a, 28b having pin receptacles 40 presented therein, and a flexible portion 30 extending between disks 28a, 28b to define an internal passage between connection members 28a, 28b within connector 24. Flexible portion 30 can be a bellows or other flexible member, such as a silicon rubber tube, including a pair of collars 38a, 38b.

Connector 24 further includes one or more conductive members 32 extending between connection members 28a, 28b to provide an electronic communication path between connection members 28a, 28b. Conduction members 32 can have an insulated portion 34 and one or more conductive portions 36, such as on ends thereof. In the embodiments described herein, the interconnection is an electrical and/or electronic interconnection, although, as described above, the system can be used to provide fluid communication and/or optical communication between functional components in the containers. As examples, if device 20 has drug delivery capability, one or more of conductors 32 can be replaced or supplemented with tubular conduits. If device has optical capability, one or more of conductors 32 can be replaced or supplemented with optical cabling. Connector 24 can further include an outer boot or sleeve 42.

Bellows 30 can be made of biocompatible material such as titanium, nitinol, stainless steel, nickel, or alloys thereof, as well as polymers such as nylon or polyurethane. For long term implantation, titanium and stainless steel bellows 30 can be used, as they do not possess the porosity of the polymeric materials, they can be weldable to adjacent bodies of similar material, and they can provide optimal hermeticity. Bellows 30 can be of any appropriate shape, but can have a shape similar in cross-section to the cross-section of container 22 to inhibit the occurrence of edges or ridges that can give rise to problems such as the formation of blood clots in the vasculature. Characteristics that can be useful for bellows 30 are described in U.S. Patent Publication No. 2006/0217779, which is incorporated herein by reference in its entirety. It should be noted that, as described above, although the present embodiment describes flexible bellows 30 for housing the interconnection system, other flexible configurations can alternatively be used, such as flexible silicone rubber tubing.

Collars 38a, 38b can be attached to each end of the bellows 30. Collars 38a, 38b can be integrally formed with bellows 30 or they can be attached to bellows 30 by welding or other techniques. Collars 38a, 38b can function to facilitate the joining of each end of bellows 30 to adjacent containers 22.

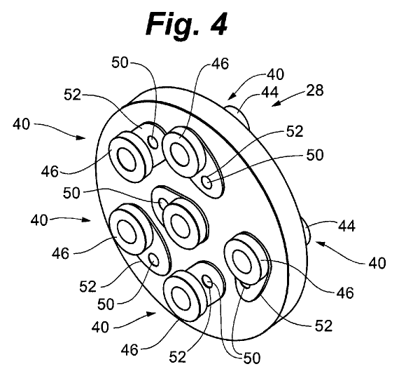

Referring to FIG. 4, disks 28a, 28b can be shaped to be press-fit into respective collars 38a, 38b at each end of bellows 30. Disks 28a, 28b can be formed out of any of a variety of printed circuit board substrate materials, such as FR4 glass epoxy substrate our other suitable substrate materials. Alternatively, disks 28a, 28b can be formed of molded plastic having conductive elements thereon or therein, and/or having circuits plated onto the plastic material. The surface of each disk 28a, 28b can include a plurality of pairs of conductively plated through-holes. Each pair can include a larger aperture (not depicted, as pin receptacles 40 are disposed therein) and a relatively smaller aperture 50. Each aperture pair is surrounded by and electrically coupled with a corresponding conductive region 52, which can be formed of copper having a solderable layer or other conductive materials.

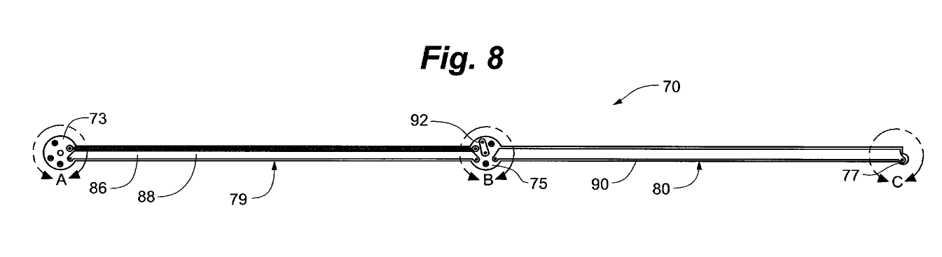

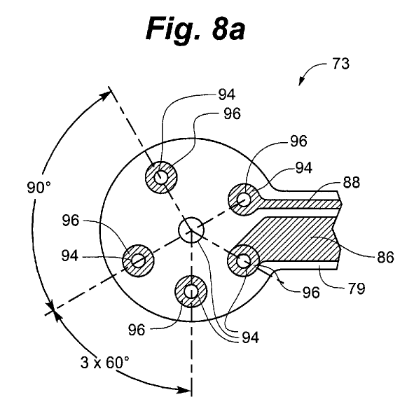

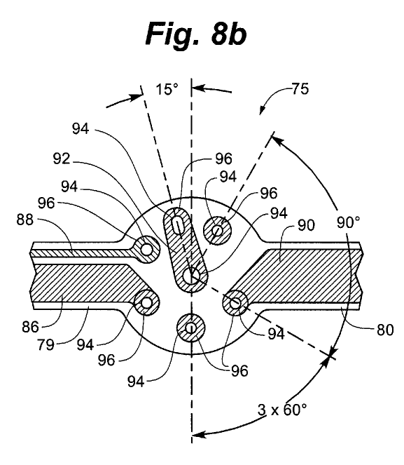

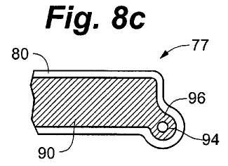

The plurality of pin receptacles 40 can be press-fit into the larger plated holes. Each pin receptacle 40 includes a conductive barrel 44 having a socket 46. A conductive clip 48 (FIGS. 8a and 8b) for engaging a lead pin is disposed within socket 46. One example of pin receptacle 40 can be a receptacle of the type manufactured by Mill-Max and proportioned to accommodate a #4 contact. Although a disk shape can be used for end members 28a, 28b to inhibit the occurrence of edges or ridges that can give rise to problems such as the formation of blood clots in the vasculature, other shapes suitable for supporting the necessary receptacles can be used.

Referring again to FIG. 3, conductors 34 can be extendable between disks 28a, 28b and provide for electrical and/or electronic communication between components in different ones of the containers. Conductors 34 can be a plurality of discrete wires or a pre-assembled cable assembly or flex circuit assembly. Conductors 34 can be fairly short in length, although longer conductors 34 can be used if needed to increase flexibility of the device or eliminate electronic cross-talk by increasing circuit-to-circuit separation.

Each conductor 34 can include exposed contacts or pins or other conductive portion 36 at its ends. The conductors 34 can be selected to be of a suitable gauge (e.g., 36 AWG) for the application for which implantable device 30 is to be used. Copper and other materials can be used, which are relatively inexpensive and good conductors, but are not otherwise biocompatible. The ability to use standard wiring and cabling within connector enables the device to be smaller, less expensive, and less susceptible to corrosion over time.

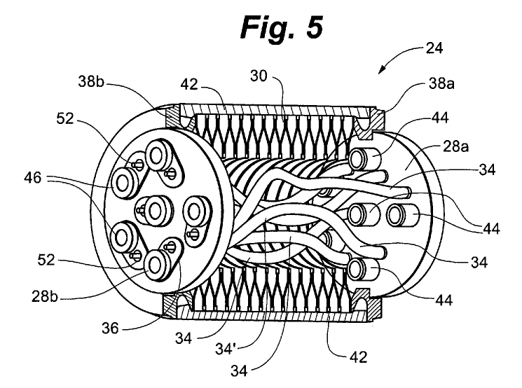

Referring to FIG. 5, if desired, one of the conductors 34 (e.g. a central conductor) can be a mechanically coupled wire to prevent over extension of bellows 30 due to excessive flexing, or excessive tension on device 20, such as when device 02 is pulled proximally for explantation from the blood vessel.

Again, although conductors 34 are depicted as conductive wiring, they can be any form of electronic or fiber optic cabling, wiring, pressure vessels, or other conduits can be used to communicatively couple the functional components of containers 30 in various ways, thus enabling for transmission of signals, power, light, and/or materials (e.g., in the case of an intravascular drug delivery device, drugs to be administered to the patient) between components housed in separate implanted containers 22.

Referring to FIGS. 3 and 5, boot or sleeve 42 can be a polymeric sleeve or overmold positionable over bellows 30. Boot 42 can function to eliminate the effects of surface irregularities (e.g., ridges, edges, and valleys) present on the outside surface of bellows 30. In particular, using boot 42 to provide a smooth outer surface on bellows 30 can minimize turbulence and clotting of the blood that could otherwise result from a rippled bellows 30 surface. Boot 42 can include an anti-thrombogenic and/or anti-proliferative coating such as those discussed above. Boot 42 can be constructed to provide rigidity and columnar strength to bellows 30, and/or to decrease the flexibility of device 20 in sections where more rigidity is desired. Also, boot 42 can be more flexible than bellows 30 such that the structure of bellows 30 alone governs the flexibility of each articulation FIG. 5 illustrates an exploded view of the assembled connector assembly 24. In this figure, bellows 30, collars 38a, 38b, and boot 42 are partially cut away to enable the interior of connector assembly 24 to be seen. One example series of steps for assembling the components is described below with reference to FIG. 5. The steps described below do not have to be performed in the described order and one or more of the steps can be eliminated or replaced with other steps without departing from the present disclosure.

If collars 38a, 38b are not provided integral with bellows 30, collars 38a, 38b can be attached to bellows 30. Pin receptacles 40 can be press-fit into the plated holes of disks 28a, 28b such that they are positioned as depicted in FIG. 4. One conductive end 36 of each conductor 32 can be electrically coupled to a first one of disks 28a. 28b, which can be carried out by soldering the un-insulated ends or pins 36 at one end of each conductor 32 into the small apertures 50 in disk 28. Disk 28 can then be press fit into collar 38, with the conductive regions 52 facing outwardly. The free ends of conductors 32 (not yet soldered or otherwise connected to disk 28) can be passed through bellows 30 such that they extend out the open end. These free ends can then be electrically coupled to the second disk 28 in a similar fashion as described above, and disk 28 can be press fit into collar 38. Flexible polymeric boot 42 can be placed over bellows 30. This can be performed before or after connector assembly 24 can be connected to containers 22.

As depicted in FIG. 5, conductors 32 can be of sufficient length to enable bellows 30 to freely flex and to inhibit potential damage to the wire 32 by device vibrations. This can relieve the strain on conductors 32 that can be caused by flexing of the connector assembly 24, and thus improve the reliability of the electrical connection formed within the assembly 24.

In an embodiment, the electrical connections can be implemented as male pins and be of different lengths to sequence the electrical connections. This can be desirable in circuit applications in order to apply power and/or ground to the circuits before the other signal connections. Additionally, where fluid communication is needed between functional components, the electrical connections can be supplemented or replaced by fluid-tight male-female connections to form flexible fluid conductors/pipes for the transport of fluids. Similarly, where optical communication is needed, optical conductors in the form of optical fibers or their equivalents can be used alone or in combination with fluid conductors and/or electrical/electronic conductors.

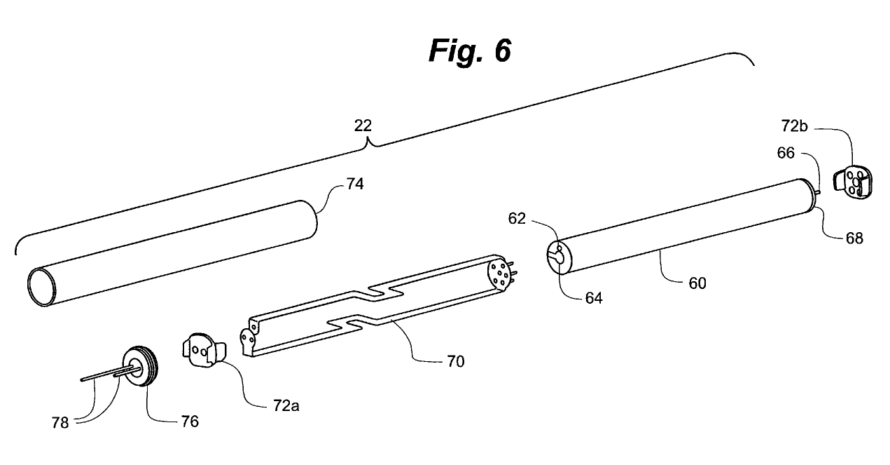

Referring to FIG. 6, an embodiment of container 22 generally includes a functional component 60 (depicted as a capacitor) having a ground pin 62 on a first end 64 thereof and a second ground pin 66 on a second end 68 thereof, a flex circuit 70, a pair of module clips 72a, 72b, and a sleeve 74. Other functional components include batteries, microprocessors, and circuitry for generating electrophysiological pulses for defibrillation, cardioversion, and/or pacing. Container 22 can further include a pin feed 76, such as a bipolar feed, having one or more pins 78 thereon. Clips 72a, 72b can help to support the contact pins 84 (described further below) in parallel to one another and enables them to be easily plugged into corresponding receptacles 40 on the connector assembly 24. Sleeve 74 can function to eliminate the effects of surface irregularities (e.g., ridges, edges, and valleys) present on the outside surface of component 60. In particular, using sleeve 74 to provide a smooth outer surface on the component 60 minimizes turbulence and clotting of the blood that could otherwise result from a rippled bellows surface. Sleeve 74 can include an anti-thrombogenic and/or anti-proliferative coating, such as those discussed above.

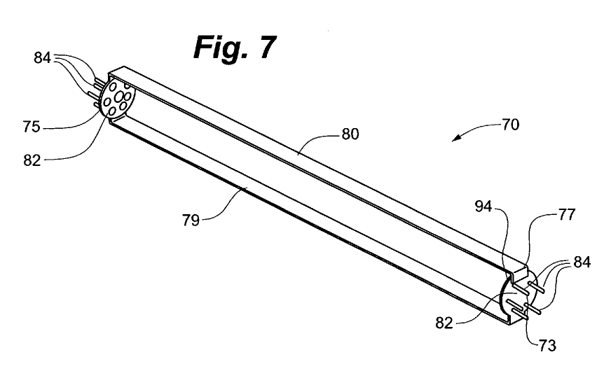

Referring to FIGS. 7 and 8, embodiments of one aspect relates to a flexible circuit 70 that can be utilized with a component 60 to enable electrical connectors, for example, in a manner that improves manufacturability and reliability. Flexible circuit 70 generally includes a first end portion 73, a middle portion 75, and a second end portion 77. Flexible circuit 70 further includes a first circuit portion 79 extending between first end and middle portions 73, 75 and a second circuit portion 80 extending between middle and second end portions 75, 77.

Referring to FIG. 7, each end portion 73, 77 can include a stiffener plate 82 for adding structural integrity to the flex circuit 70 at the ends 73, 77 thereof. In addition, each end portion 73, 77 can include one or more pins 84 operably and/or electrically coupled thereto. Stiffener plates 82 can also help clips 72a, 72b to support the contact pins 84 in parallel to one another and enables contact pins 84 to be easily plugged into corresponding receptacles 40 on connector assembly 24.

Referring to FIG. 8, first circuit portion 79 includes a first circuit pattern 86 and a second circuit pattern 88, each extending into first end and middle portions 73, 75. Second circuit portion 80 includes a third circuit pattern 90 extending into the middle and second end portions 75, 77. As described below, middle portion 75 can include a fourth circuit pattern 92 thereon between two apertures 94 on middle portion 75. Circuit patterns 86, 88, 90 can be governed by the arrangement of components within containers 22 and can vary depending on the functional components being used. Such variations in circuit patterns and configurations are within the scope of the present disclosure.

Referring to FIG. 8a, in an embodiment, first end portion 73 can be circular in shape to match the profile of disks 28. First end portion 73 includes a plurality of apertures 94 for insertion of contact pins 84 therethrough. Each aperture 94 can include a circuit pattern 96 presented thereabout, which can be connected to one or more of the circuit patterns on first and second circuit portions 79, 80 of flex circuit 70. For example, as depicted in FIG. 8a, circuit pattern 96 presented with one of the apertures 94 is connected to first circuit pattern 86 and another to second circuit pattern 88 on first circuit portion 79. Again, the circuit patterns and apertures can be governed by the arrangement of components within the container and can vary depending on the functional components being used.

Referring to FIG. 8b, middle portion 75 can also be circular in shape to match the profile of disks 28. Middle portion 75 includes a plurality of apertures 94 for insertion of contact pins 84 therethrough. Each aperture 94 can include a circuit pattern 96 presented thereabout, which can be connected to one or more of the circuit patterns on first and second circuit portions 79, 80 of flex circuit 70. For example, as depicted in FIG. 8b, circuit pattern 96 with one of the apertures 94 is connected to first circuit pattern 86 and another to second circuit pattern 88 on first circuit portion 79. The circuit pattern 96 presented with another of the apertures 94 is connected to third circuit pattern 90 on second circuit portion 80. Further, a circuit pattern 92 is presented as the circuit patterns 96 with two of the apertures 94 can be connected to each other.

Referring to FIG. 8c, second end portion 77 includes an aperture 94 for insertion of a contact pin therethrough. Second end portion 94 can include a circuit pattern 96 presented about aperture 94, which can be connected to third circuit pattern 96 on second circuit portion 80.

Referring again to FIG. 7, in an embodiment, flex circuit 70 can be assembled by first folding or otherwise bending or crimping first and second circuit portions 79, 80 proximate middle portion 75. Also, first and second circuit portions 79, 80 can also be crimped proximate first and second end portions 73, 77. Aperture 94 included in second end portion 77 can be placed over pin 84 disposed in aperture 94 included in first end portion 73 so as to capture second end portion 77 thereon. In this configuration, flex circuit 70 includes two generally disk-shaped end portions 73, 77 with first and second circuit portions 79, 80 extending therebetween. Pins 84 can be presented in apertures 94 included in first end and middle portions 73, 75 such that pins 84 can be outwardly oriented relative to flex circuit assembly 70.

Referring again to FIG. 6 to view container 22 parts, to assembly container 22, flex circuit 70 can be coupled with component 60 such that first and second circuit portions 79, 80 extend along a length thereof and clips 72a, 72b can be coupled at first and second ends 64, 68 of component 60. Bipolar feed 76 can be coupled to first clip 72a. Sleeve 74 can then be placed over component 60 and flex circuit 70 assembly.

Assembly of device 20 can begin with container 22 assembled such that the functional components (e.g., battery, or capacitor, and/or other components) are housed and electrically coupled via flex circuit 70 or other conductors to contact pins 84. Contact pins 84 can extend through plastic connector or clip 72 that can help to support contact pins 84 in parallel to one another and enables them to be easily plugged into corresponding receptacles 40 on connector assembly 24. The pattern formed by pins 84 (and the corresponding receptacle 40 pattern on corresponding connector assembly 24) can be governed by the arrangement of components within container 22 and can vary depending on the functional components being used. Connector assembly 24 also can be pre-assembled at this stage, although positioning of boot 42 can be delayed until later in the assembly process.

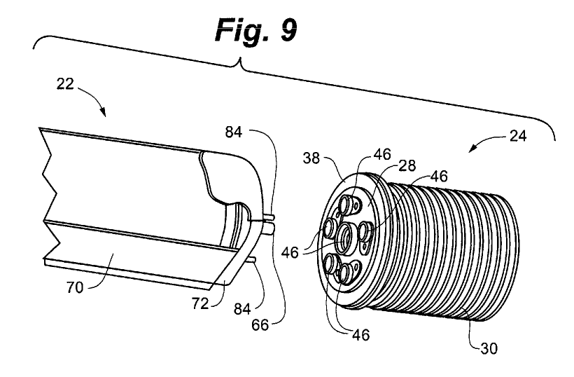

FIG. 9 illustrates container 22 and assembled connector assembly 24 being moved into engagement with one another as part of the process of assembling device 20 as depicted in FIG. 2. As discussed previously, this step can be repeated multiple times to form device 20 using multiple containers 22 interconnected by multiple connector assemblies 24. As container 22 and connector assembly 24 are brought together, contact pins 84 and second ground pin 66 can be inserted into sockets 46 of receptacles 40. Receptacles 40 electrically couple contact pins 84 and thus flex circuit 70 to conductors 34 within connector assembly 24, enabling for electrical conduction and/or communication of signals between the various containers 22 forming a complete device 20.

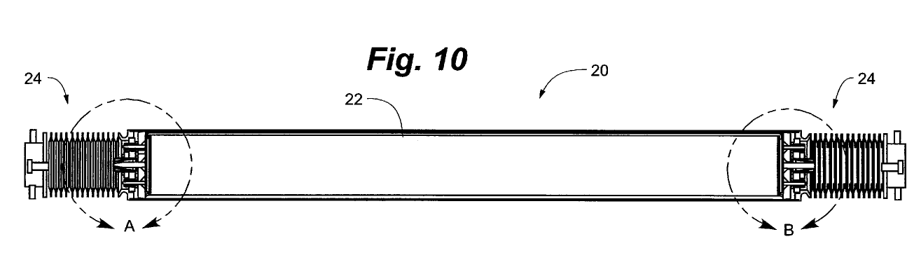

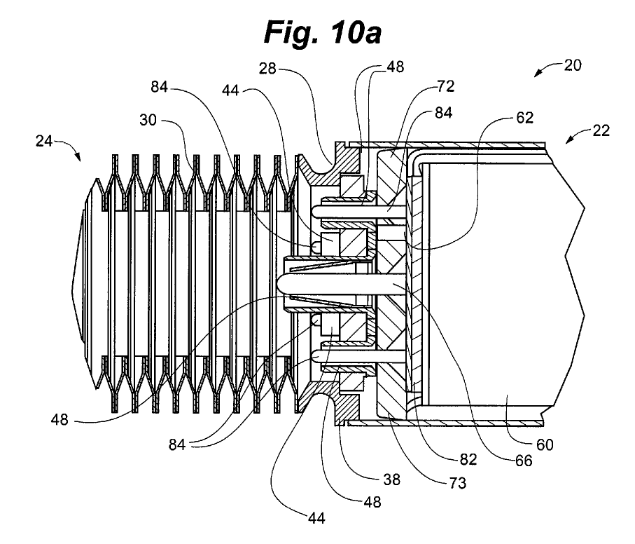

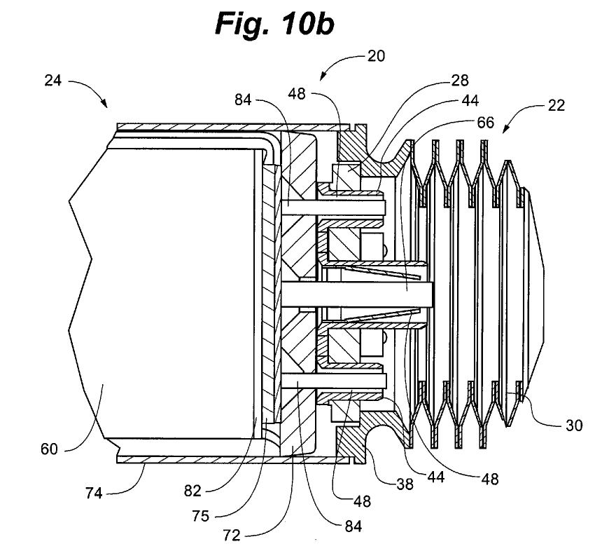

FIGS. 10 illustrates an alternate embodiment of container 22 with assembled connector assemblies 24 at each of the pair of end portions of container 22 in which like reference numerals denote similar components as has been described. FIGS. 10a and 10b show cross-sectional cut-away detail of the manner in which the contact pins 84 and ground pin 66 engage conductive clips 48 in corresponding sockets 46 of receptacle 40.

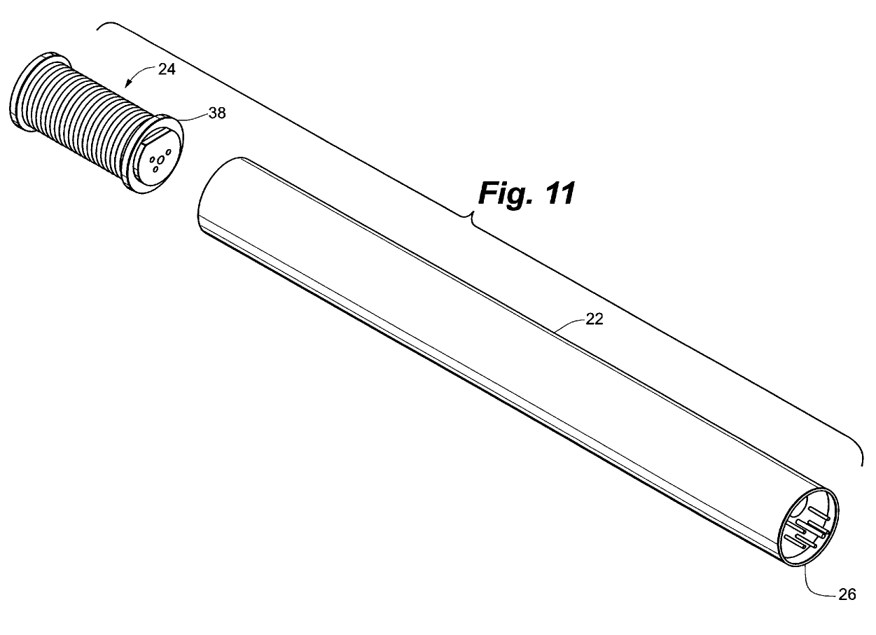

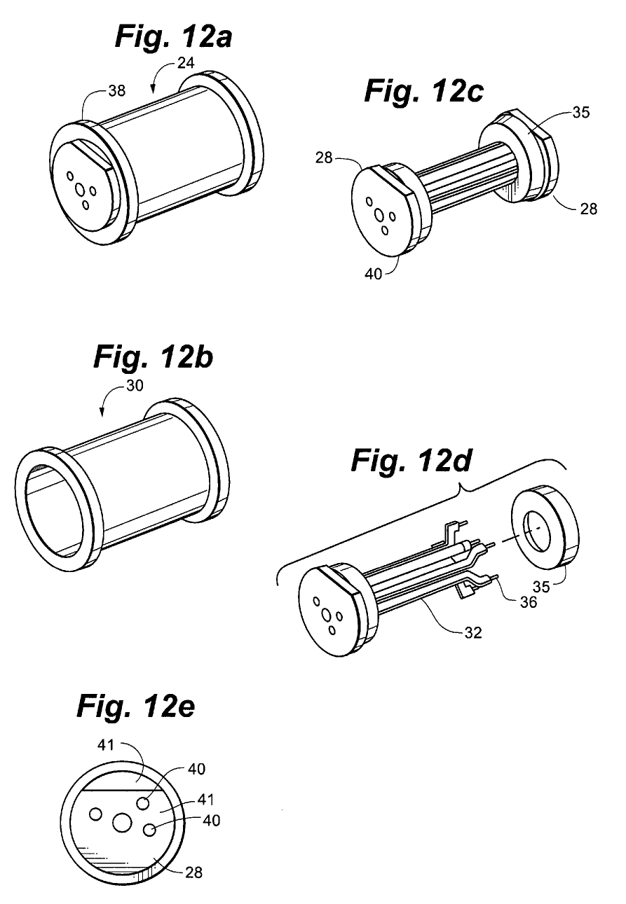

FIG. 11 shows another embodiment of a container 22 and a connector assembly 24 in which a tubular lip 26 of container 22 is integrally extended as an end cap that mates with a corresponding collar 38 of connector assembly 24. FIG. 12a shows a detail view of an assembled connector assembly 24 in accordance with this embodiment. In this embodiment, at least a portion of the exposed surface of collar 38 is adhered to a corresponding surface of the end portion of container 22 such as the clip 72 using a UV curing adhesive. In this embodiment, as shown in FIGS. 12c and 12e female connector disc 28 in the form a printed circuit board includes apertures 40 for mating with corresponding contact pins 84 and ground pin 66, as well as apertures 41 which are adapted to receive pinned ends 36 of conductors 32. The printed circuit board includes internal connections between corresponding ones of apertures 40 and 41, for example. To facilitate easier manufacturing of this embodiment of connector assembly 24, conductors 34 are retained by a pair of dielectric cups 35 and the pinned ends 36 of connectors 32 are pre-bent at angles corresponding to the respective apertures 41. Solder, mechanical connections and/or conductive adhesive may be used to secure the ends 36 of connectors 32 in the corresponding apertures 41. As shown in FIGS. 12c and 12d, the dielectric cups 35 are positioned in place at the inside ends of connector assembly 24 proximate the corresponding collars 38 and serve to provide additional structural support and insulative protection for the connections of conductors 32 within connector assembly 24.

A connecting process, such as welding, can be used to form a continuous, hermetic seal between connectors 24 and containers 22 after connectors 24 and containers 22 are electrically coupled when contact pins 84 are inserted into receptacles 40. Many types of welding, using lasers or e-beams, for example, can be used to create a circular weld, depending upon the necessary heating and weld thickness. The welding device can form the weld by rotating about the seam between the connector 24 and container 22. Although all or a portion of the implant 20 can be encapsulated in an elastomer, the resultant weld can enclose the interiors of the connectors 24 and containers 22 such that an elastomer is not needed to seal the components 60 and conductors 34 within device 20 from the bloodstream. If desired, the device 10 can then be coated using coatings of the type referenced above. Alternatively, a continuous hermetic seal between connectors 24 and containers 22 can be created by an elastomeric/polymeric coating, applying, or spraying process.

Use of flexible connector assembly 24 to form device 20 can provide a number of advantages. For example, use of connector assemblies 24 can enable simplified assembly of device 20 in a modular fashion. Thus, rather than using an assembly process requiring one to thread various segments or containers of device over a longer cable, flex circuit, or thread/wire extending the through multiple segments or containers of device, the different segments or containers 22 can simply be plugged into one another. In addition to simplifying assembly, this method can reduce material costs, as the use of elongate cables, flex circuits, or the like extending through devices can necessitate the use of service loops (additional lengths of the cable material coiled at various points within the device) as in previous systems can be eliminated or minimized using the disclosed assembly method.

Moreover, the modularity of device 20 can be beneficial in that a change to the design of one of the components within container 22 will be less likely to require corresponding changes to other components 22 or flex circuits 70 in device 20. For example, in prior systems if an improvement in the design of component leads to the shortening of one or more individual containers, flex circuit or other component extending through multiple ones of the containers can have to be redesigned to accommodate the change in length. In the described embodiments using connector assembly 24, downstream modifications of that nature can be avoided.

Intravascular device 20 of the type described herein are adaptable for use in a variety of applications, including single chamber atrial or ventricular pacing, dual chamber (atrial and ventricular) pacing, bi-atrial pacing for the suppression of atrial fibrillation, bi-ventricular pacing for heart failure patients, cardioversion for ventricular tachycardia, ventricular defibrillation for ventricular fibrillation, and atrial defibrillation. Device 20 can be adapted to perform multiple functions for use in combinations of these applications. Device 20 can be implanted for permanent use, or it can be implanted for temporary use until more permanent interventions can be used. Examples of applications for the intravascular device 20 can of the type described herein are described, for example, in U.S. Patent Publication Nos. 2006/0217779 and 2005/0043765, which are both incorporated by reference in their entirety.

Many of the device configurations, components, retention devices and methods, implantation methods and other features are equally suitable for use with other forms of intravascular implants. Such implants can include, for example, implantable neurostimulators, artificial pancreas implants, diagnostic implants with sensors that gather data such as properties of the patient's blood (e.g. blood glucose level) and/or devices that deliver drugs or other therapies into the blood from within a blood vessel.

More particularly, fully implantable intravascular systems can be used for administering drugs including hormones, chemotherapeutic agents, pharmaceuticals, synthetic, recombinant or natural biologics, and other agents within the body. Generally speaking, the systems include drug reservoirs and associated components (e.g. batteries, electronics, motors, pumps, circuitry, telemetric components, and sensors) that are anchored in the vasculature and programmed to administer drugs into the bloodstream or directly into certain organs or tissues. Drug delivery microtubules can extend from the device body and into surrounding vessels in a similar way that the leads in the embodiments described above extend from the device body. These microtubules can be positioned within the vasculature to deliver drugs directly into the bloodstream, and/or they can extend from the device through the vascular into or near a body organ. For example, by directing drugs to a particular aortic branch (e.g. hepatic artery, renal artery, etc), an intravascular delivery device can achieve target delivery of therapeutic drugs to specific organs including the brain, liver, kidneys etc. In some embodiments, such intravascular drug delivery systems can be controlled remotely using telemetry or via internal intelligence that can be responsive to in-situ sensing of biological, physical or biochemical parameters.

Also, although the embodiments have been described in the context of intravascular implants, alternative embodiments can be used to house medical devices implanted elsewhere in the body, including subcutaneous pockets, body organs, or other body cavities.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A modular implantable medical device, including:
first and second component containers;
a flexible connector that operably connects the first and second containers, the connector including a pair of end members and a flexible portion extending therebetween to define an internal passage between the end members, wherein one of the end members includes a first receptacle configured to communicate with a portion of the first container and the other of the end members includes a second receptacle configured to communicate with a portion of the second container; and
a plurality of conduits extending between the end members in the internal passage to provide a plurality of communication paths between the first and second containers, such that when the first container is in communication with the first receptacle and the second container is in communication with the second receptacle, a portion of the first and second containers are in communication via the plurality of conduits.

2. The device of claim 1, wherein the flexible portion includes bellows and the pair of end members includes collars to which the bellows are attached.

3. The device of claim 2, wherein the flexible portion includes a boot presented over the bellows.

4. The device of claim 1, wherein the conduit includes a conductor to provide an electrical communication path between the first and second containers.

5. The device of claim 1, wherein the conduit includes a fluid conduit to provide a fluidic communication path between the first and second containers.

6. The device of claim 1, wherein the conduit includes an optical cable to provide an optical communication path between the first and second containers.

7. The device of claim 1, wherein the first container includes a first male member at an end thereof and the second container includes a second male member at an end thereof, the first receptacle configured to receive the first male member and the second receptacle configured to receive the second male member to provide the communication path between the first and second containers.

8. The device of claim 7, wherein the first and second male members include electrical contact pins.

9. The device of claim 1, wherein the first container includes a first clip and a first plurality of electrical contact pins at an end thereof and the second container includes a second clip and a second plurality of electrical contact pins at an end thereof, the first and second clips supporting individual pins in the first and second plurality of pins, respectively, in a substantially parallel relationship to one another.

10. The device of claim 4, wherein at least one of the first and second containers includes a functional component and a flex circuit therein.

11. The device of claim 10, wherein the flex circuit includes at least a pair of connector portions and at least one elongated portion, each connector portion including a plurality of connectors operably coupled to a respective one of the first and second receptacles.

12. The device of claim 11, wherein the flex circuit includes two elongated portions and three connector portions, with a connector portion between the two elongated portions and a connector portion at each other end of the two elongated portions, wherein the flex circuit is formed in a generally tubular configuration.

13. The device of claim 1, wherein the first and second compartment containers are generally elongated and cylindrical and the first and second receptacles are generally circular.

14. A flexible connector for an elongate medical device, the connector including:
first and second end members, each of the end members formed of non-conductive material;
a flexible portion extending between the end members defining an internal passage therebetween; and
a conductive member presented in the internal passage extending between a first conductive region presented on the first end member and a second conductive region presented on the second end member to electrically connect the first and second conductive regions.

15. The connector of claim 14, wherein the flexible portion includes bellows.

16. The connector of claim 14, wherein the connector operably connecting first and second component containers, the first container including a first electrical contact pin at an end thereof and the second container including a second electrical contact pin at an end thereof, the first conductive region configured to operably receive the first pin and the second conductive region configured to operably receive the second pin to provide an electrical communication path between the first and second containers.

17. The connector of claim 16, wherein a first receptacle is provided with the first conductive region for receiving the first pin and a second receptacle is provided with the second conductive region for receiving the second pin.

18. The connector of claim 14, wherein the connector operably connecting first and second component containers, the first container including a first clip and a first plurality of pins at an end thereof and the second container including a second clip and a second plurality of pins at an end thereof, the first and second clips supporting individual pins in the first and second plurality of pins, respectively, in a substantially parallel relationship to one another.

19. A method for connecting containers of an implantable medical device, the method including:
providing a first component container and a second component container;
providing a connector including a first end member having a first receptacle therewith and a second end member having a second receptacle therewith and a flexible portion extending between the first and second end members to define a passage between the end members, wherein a conduit extends between the end members in the passage to provide a communication path between the first and second end members; and
coupling the first component container with the first end member and the second component container with the second end member, such that the first and second component containers are in communication via the conduit.

20. The method of claim 19, wherein the first container includes a first male member at an end thereof and the second container includes a second male member at an end thereof, further including operably inserting the first male member in the first receptacle and operably inserting the second male member into the second receptacle to provide the communication path between the first and second containers.

21. The method of claim 16, further including operably welding the first end member to the first container and the second end member to the second container after the first and second component containers are operably coupled to the first and second end members.

22. A modular implantable medical device, including:
at least first, second and third elongated modular segments, each modular segment having a pair of end portions corresponding to opposite ends of a long axis of the modular segment; and
at least a pair of flexible connectors, each flexible connector connecting an adjacent pair of the at least first, second and third modular segments and including a pair of end members and a flexible portion extending therebetween, each end members configured to mate with an end portion of one of the adjacent pair of modular segments and including a plurality of connectors, the plurality of connectors of each end member being operably connected to corresponding connectors of the other end member through the flexible portion.

23. The device of claim 22, wherein the end portions of each modular segments are at least electrically connected to each other by a flex circuit that connects to at least one connector that mates with a connector of an end member.

24. The device of claim 23, wherein the flex circuit includes two elongated portions and three connector portions, with a connector portion between the two elongated portions and a connector portion at each other end of the two elongated portions, wherein the flex circuit is formed in a generally tubular configuration having opposed ends with the connector portion between the two elongated portions corresponding to one end of the tubular configuration and at least a portion of the connector portions at each end overlapping on the other end of the tubular configuration.

25. The device of claim 24, wherein the first, second and third elongated modular segments are generally cylindrical and isodiametric and the end portions and end members are generally circular and isodiametric.

26. The device of claim 24, wherein each opposed end includes a stiffener plate operably supporting the corresponding connector portions of the flex circuit and the connector portions include apertures and corresponding engagement structures to mate with pins extending from the end portion of a modular segment.
